SEQUENCE LISTING (24 sequences)

SEQ ID NO: 1 - protein, 780 aa, Homo sapiens
HSTGSSAAGAPLGRASGPRRPSVLPSAALSAGARRRLCPGPAALAGRPVRAADPEEPRCGWPREVKFWARASTPPRLPPSFRPLAAPASFPQISDRPFAPPPRPPPVLRSPPLGSPAAGGGAGVTMAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDMHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 2 - DNA, 587 bp, Homo sapiens
cagtagcatgctgataccaacgtttaaactatggatacatatttgaattccaaatttttcttcaaataatgtgattagagattcaaccaggaatagacaccgaaagaaaactttgcccaaataagctttctggtatttcataagcaagagatttaagttttccatttaagaagcaattgtgaattttacaacaataaaaaatgcaagtggatattgaacagtctctgctctgataattctaaatacagtacagttcacgcccctcacaagacactgaacatgtgggtcaccggcgagacagtgtggcaatattttccctgtaatgtaccaagtcttgccaaagcagtgaacattatgacacaactttttgtcacagctggctcctaataggacagtgccagccaattcaagcccagtcctttctgtgtttattcccatctctcccaaatatttggaaactgatgtcttgactcatgggtgtattcacaaattctgttacttcaagtctttttcttttaacggattgatcttttgctagatagagacaaaatatcagtgtgaattacagcaaacccctattccatgct

SEQ ID NO: 3 - DNA, 261 bp, Homo sapiens
caaataatgtgattagagattcaaccaggaatagacaccgaaagaaaactttgcccaaataagctttctggtatttcataagcaagagatttaagttttccatttaagaagcaattgtgaattttacaacaataaaaaatgcaagtggatattgaacagtctctgctctgataattctaaatacagtacagttcacgcccctcacaagacactgaacatgtgggtcaccggcgagacagtgtggcaatattttccctgtaa

SEQ ID NO: 4 - DNA, 40 bp, Homo sapiens
gacactgaacatgtgggtcaccggcgagacagtgtggcaa

SEQ ID NO: 5 - DNA, 7 bp, Homo sapiens
  misc_feature (2)..(2): n=G or A
tnttnnn

SEQ ID NO: 6 - DNA, 8 bp, Homo sapiens
ttgtttac

SEQ ID NO: 7 - DNA, 2776 bp, Homo sapiens
cacgcggtggcggccgctctagaactagtggatcccccgggctgcaggaattcgaggctgcagtgaactatgattgcaccactgcactccagcctgggtgagagagcaataccttgtctcaaaacaaacaaacaaacaaaaccccatgagatatcacttcataccctttaggttggctaaaataaaaaagactataacaagtgttgacaaggatgtggaaaaactggaaccctgacacattgctggtgggattgtaaaatggtgtgcccactttggaaaacagactggcagttcctcaaaaacaccgagttacgttatgatcctgcagttctgtccctaggtatatactcaagagaaataaaaatatatgtccacaagtaaccttgtacatgaatgctcacagcagcattattcataatagcccataaaagtagaaacaacctaaatattcatcaattcatgggatgaataaacaaaatgtggtatatgtgtataatggaatattgaccataaaaaggaatgaaatattaatataagctataacatggatgagcctccacaaatactatgctaagtgaaagaagaaagtcacaaaggacttcatattctatgattctatttatatgaattgtccagaataggtaaatctatagagaaagaatatctctatctagagttggtggaatgactgttaatggagagggggttcctttttggagtgatgaaaatgttctaagggtagatttggtgatgatggcacaactctgtcaataaactaaaactcattgaactgtacattttatttatttatttttgagatggagtcttgctctggggctgaagtgcagtggcgcaatctcggcttgtaacctctgcctcccagggtcaagcgattctactgcctcagccccccgagtagctgagattacaggcacgtgccaccacgcccagctaatttttgtatttcttagtagagatggagtttcaccatgttggccaggctggtcttgaactcccggcctcaagtgatccacctgcctcggcctcccaaagtgctgggattacaggcgtgagctgccatacccggcctgaattgtacattttacttctatggtatttacattttagattatattaattattcctcaataaagctgtgattttaaaaagcaggctaggcgcagtggctggtgcctataatcccagcactttggaaagctgaggcaggaggatcacttgagcccaggagtttcagactagtctaggcaacatgtcaagacacagtctctactaaacaattaaaattaaaaaaaaaaattagccaggcatggtggtgtgcacctgtagtcccagctacttgggagcctggggtgggaggattccttgagcccgggaagtcgaggctgaagtgagccgtgattgcgccacagcactccagcctgggcgacacagcaacaccctgtctcatggaagaaagaaagaaaagaaaggaagaaagaaaaaaaaaaagcagattggaactctggaattaacaagaagtaggacgcacggagcacttccgcctgagtggagactgtggatccgggtcaacctgactacctaaatcacaggccaataaatggtctttcagtggtcagtccctgtaagatccgtggctctcagcttcttatcttaggggctgtggaggaaggacatgattatgttgatttaagcgctgaatattttcccttgtgatacccatcctcgcaaaactttgcttcaaccacaaacgaggaccttctgtaccagaggggcaataacacaatgaagctaggaagaaatgcagagcaccccagcatacagtccataagcttcctgaagtggggggcctcaggcatcgctgcctccccaaagaggatcaggcccagaacagtatgctccagaaataagactggaaaaagggaaagaggggcctcaagtccaggagaccagcggctttctgaacgcgcacctgccaacccactttggacaggtcacgatggacagcgtggcaggaaaagaaaaggtcactgtctacccaacacatgagaaactgtttctcgtgcctcacgtccccactccgtccccacccatgttgtctgagtccctcggtgtcagaaacactgctaagaaatttaagaaattctgttaatgagtttaagaaatgtttttaatgattaaaagtcagtgacttgtgaataaccatgtaacttacaaacgcaaggaactctgaaagtgtgcagcaccaccgatcagaagagaaaaccaagggacccgaaatatgctttaattaaattttcttttaaaatgtcactggaaagaacatcttgggaagacggcctggccgatcgccgtgtgaagggcaagccactctggccgagagggagccccacacctcggtctccccagaccggccctggccgggggcatccccctaaacttcggatccctcctcggaaatgggaccctctctgggccgcctcccagcggtggtggcgaggagcaaacgacaccaggtagctgccgcggggcagagagtggacgcgggaaagccggtggctcccgccgtgggccctactgtgcgcgggcggcggccgagcccgggccgctccctcccagtggtggggctgctgcccctgcccctgcccctgcccccacccccacccccacccccacccccagccggcgcccgcgcccgcccccgcgccgggcccggctcggcccgacccggttccgccgcgggcaggcggggcccagc

SEQ ID NO: 8 - DNA, 1664 bp, Homo sapiens
agcttggatgtggctgcctgcacaccccacgagatatgcaggatattacgtgtacaggtcaccctataaactctgaaacaacataaatgatagctattgctggctaacatgtgcaagagaaggtgaggttgccgtgtgccagtgggaaggatatgtgggtgtctggagggtggggcaaagttcaccataggtgcctggctaatcaacaagggtgagtttaagagaagtttgtgaaagggccagttcccgcctgatctccagttcccctgcctaatgagatctgggtttgctttccggagtgggtcctcctgggcgcccgctgccacaaatagcacagtggcaggcacagctggcagcggagggcaggcagtggagcgggcatagcacacattgcgttcccgaggaggggagccctgatgtgccggcacccaggggctttgtgcattccgctccggcagctcgagcgtcacggcccgaacaaagcggctttgcagggcgctctcctactcctcgccattggccaccgggccgcgacctctccttgctctggcagagtcccagcgccttgcgtaggcacaaagtcagcccctcacccccagcgcatgaagtaggggcgggcatagctcttgggccgctggagaggggtggggttgtggggtcggagctggcggcgcctccccgagcccgtccgcgttgcccttcaccctcctcgttcccccgcccaccacaccgccctggcacctcccgccacctgtttccttgtcctcccagctcgccttccccttctccttattttgcatcctggggttccagggacaaggtccctcccgggccgcctcccaccctacgcacttctgagcctcaagggcacccggtcccgggtcccgggtctagaccggctcatcgcacagagtagcagagccgggctcatcgaggaggcaggaggggctcgccagcgtggcacgggcgcccggcgggaacctccacccgccccgcggccgcgcgtccccgcctcgaattcagccccgccccggtgcgccgggctggaggggcgctgacgctcagcggtgtcccatcggtgaccttggacggtccctccacctctccggcctcagtttcccttggctgcagcggccgcggggcgctaggtgggagccgctgagcgctcccggggccccgcccaccgcgagcagccaatcgggcgccgccctccggggggtgtgtcccggggccgaggcccggggcccggagggcgcgcggggcgggcggggcttccgggtcgggcctcgggacactggctcgcgcggaccggggcagggggcgggccgaggggcggtgcgtgtcgcgggggcgcggctggcacggacgcgcggaggcggcgccgggcatgggccgtggacgcggcggccccgcggcgggggcagcgggcggcgggggcggaggcggccgctagcgccctgcccggcgccgcctccttcggcgttcgccccacggaccggcaggcggcggaccgcggcccaggtgcccgggggcgggcgggcgggcgggcgggaacgcgcgcgggggtccgcggtccgggcttcccaggtcccgggacccggagggcggcggacgggggagggcaggggctgggcggcgcatgcgcggggcccggggtctcgggg

SEQ ID NO: 9 - DNA, 1147 bp, Homo sapiens
tgttctgcaaccagtcttgtcctttaaatacttgtactgtatacaggctctttttcataggtccattacttaaaatgatgtaagtgtgtttttggtggcaggggggtgggagttgtttgttttgttttgttgagacacggtcttactctgtcacccaggctggagtgcagtggtgtgatcttggctcactcctggcctcaagtgatccacccacctcagcctcctaagtagctgggaccacaggtgtgtaccaccacacccagctaattttttttttttttttttttttttttgtagggacggggttttgtcatatcacccaggctggtctcaaactcctggactcaagggatcagcctgtctcagcctcccaaagtgctgggattacaggtgtgagccactgcaccggtcctgatttgagtttttgtaagacagggaacaatgttcagaatttagcaccaatgtcagactcattctgtaaatttttattgaacgtctgcctggtgtaggagaggaagatgacagacaagaattcttcctccaagagttacaggtcagttgagcagaaaaggcatacatcaatacccacaatgagagttgtcgtgattcagaggagggacaaagtccttcccctggagggatcctgagcactttggagaggaaaggcatctgtactgccccccaaatgtgtagaatgggatgcattcctggcagaaagaagtaggataaagtacagaggccagggctgggtgcagtggttcacgcctgtaatcccagcactttgggaggccgagacagcagatcacctgaggtcaggagttcgagaccagcctggtcaacatggcaaaaccctctctctactaaaaatacaaaaattagccaggcacaatggcaggtacctgtaatcccagctacttgggaggctgaggcaggagaattgcttgagcccaggaggcagagatcgcagtgagccaagactgcgccactgcactccagcctgggcaacagagcaagactctgtctcataaaaaaagaaaaaaaaaaagtacagagtccaggaagcctggggtggggctggcagatgccgagtcatctattttggccagagttcaaggcttgctaggggacatgaagagaagattcgtgc

SEQ ID NO: 10 - DNA, 1366 bp, Homo sapiens
ccagtcattaatctgttgcccatttctgtcattttatgaatgtcacataggccgggcgcggtggctcacgcctgtaatcccagcactttgggaggccgaggcaggcggatcacgaggtcaggagatcgagaccatcctggctaacatggtgaaaccccatctctactaaaaaatacaaaaaattagccaagcgtggtggcgggcgcctgtagtcccagctactcgggaggctgaggcaggagaatggtgtggacccgggagacggagcttgtagtgagctgaaatcacaccactgcactccagactgggtgacaaagcgagactccatcttaaaaaaaaaaaagaatgtcacataatgaatcatatggcatataaccgtttgagactcagggtaattctcatgagactcatccagcttgttggtgcatcaacagtttattcctttttattgctgagtaatttccatggtatggaggaaccatggtttaactattcacccattggaggacatctaggttgtttccagcttggagttattatgaataaagctgctgtgaacatttgtgtacaggtttcttggttttctggtttgttttaaacagttctagccaggcacggtggctcacacctgtaatcctaacacttggaaggctgaggtaggaggactgcttgatcctaggaggcagaggttgcaaggagccgaaattgtgccactgtactccagcctgggcaacatagcaagaccctgtcattcataggtaggtggatggatggatggacggacggacagatagataggtagaaatgtaaattacagggctacgctcagtggctcatgcctgtaatctcagcactttgggaggcgaaggcgggcggatcaccagaggtcagcagtttgagaccagcctggccaacatggcaaaaccccatctctactaaaaatacaaaaattagccaagcatgctggcatgtgcctgcaatcccagctactttggaggctgaggcaggagaatcacttgaacccaggaggcggaggttacaatgagccaagatcatgccactgcactccagcctgggccacagagtgagactccgtatcagtactttctttttattgtttttctgttattatagtttaagttcattgttattagattatatactctgtatggcttcaattcttttaaatttgttgaggtttgtttaatggtcaaagacatggtctgtctaggtgaatgttccatgggcttttagggaaaaaagtatattctagtgttgttgaatggtgtcttagtccattcaagctgctataacaaaataccgtaaactgggtgatttataaacaacagaaatttttctctcacag

SEQ ID NO: 11 - DNA, 938 bp, Homo sapiens
ctgcgcagtcagtgatgactgagagattcctggccccgtgggctatggctccttcaacagtttgttgtttaaaggttcttcactttctcagcgtgctgatcaagagacaagcctggaggagaggctcagtggtgctcctgtgtagatgatgaattcaggtgtatcttggatggtaaatgacgttgcatttaaaaccaagcaagtggccaggcgcagtggctcacacctgtgatcaaagcactttagaaggccgaggcgggcggatcacctgaagtcaggagtttgagaccagcctggccagcatggtaaaaacccgtctctactaataatacaaaaaaactagctgggcgtggtggcgggcacctgtaatcccaaccactcagaaggctgaggcaggagaattgcttgaacccgggaggtggaggttgcagtgagctgagatcgcaccactgtactccagcctgggcgacaagagcaagactctatctcaaaaataaaaaaaattaaaaattaaaatttaaaattaaaacaaacagccggacgcagtggctcactcctgtaatcgcagcactttgcgaggctgaggcgagcggaatacgagctcaggagatcgagaccaccctggctaacacagtgaaacccgtctctactaaaaaaaaaaaatacaaaaaattagccaggcgtggtggcaggcgcctgtagtcccagctactcaggagactgaggcaggagaatggtgtgaacccgggaggcggagcttgcagtgagccgagattgtgcccctgcactccagcctgggcaacagactgagactctgtctcaaaaaaaaaaaaaaaaaagaataaataaataaataataaaaaataaaaacaaacaagtgaacgttgttatacgtcagtcttaccaattgttcctctttcctcccagtagcttggagc

SEQ ID NO: 12 - DNA, 816 bp, Homo sapiens
ctacggaaggtgtggccgaaggagaggggaggggtcatctttaatgatgatggaggggaggcattggtcatatacctgagagtcaagctttgttcatcgtcagatgaaagccaacttcttccaccagattgcctcccagctgctaggtagtttcctgtggtacatcttaagcggtaggttgggataataggtcccttccaggtcttaaagatgtggattaataagaacagaattttctacaacaataaggaggatgtcccctattttaggtgaataagcgtattgcataagcacatgggaagaaaggttaagaactctaagaccgtaaatttccagttgtagaagcctcttgaaggcggaaagcccttttttgaaaattataggcgatatgatctcctccagtggacctagcactgggttaagagtctctccagacttgtcactgcaagtcacgtaccttttctaggactaagtttcttcactggaagatgaggaggttgcactagatgacctagaaattcccttccagctttaaattccctcttgtgtcacttgctggacttgttctactggacctggcgctcccctgctcctcccttagctgctacgtccgcatcccgcaccagagggcgccacaggctggcccggggcagcgtgcggtgggcggagaggcagaattaggggagtctccaggaggcgtggtgattggccgccgccgggcggaagggggcgtggggagggaaaggccgagcagcgcggtgacgtctcgctggcgggggcggggccgggccgcggagcgtgtgacgctgcggccgc

SEQ ID NO: 13 - protein, 81 aa, Homo sapiens
WGNLSYADLITKAIESSAEKRLTLSQIYEWMVKSVPYFKDKGDSNSSAGWKNSIRHNLSLHSKFIRVQNEGTGKSSWWMLN

SEQ ID NO: 14 - DNA, 21 bp, Artificial Sequence
  Synthetic
cgacgaggcccagagcaagag

SEQ ID NO: 15 - DNA, 27 bp, Artificial Sequence
  Synthetic
cgtcaggcagctcatagctctccaggg

SEQ ID NO: 16 - DNA, 20 bp, Artificial Sequence
  Synthetic
ggataagggtgacagcaaca

SEQ ID NO: 17 - DNA, 20 bp, Artificial Sequence
  Synthetic
tctgcacacgaatgaacttg

SEQ ID NO: 18 - DNA, 20 bp, Artificial Sequence
  Synthetic
tctctccgtaatggaagacc

SEQ ID NO: 19 - DNA, 18 bp, Artificial Sequence
  Synthetic
gcattatgagcatcccca

SEQ ID NO: 20 - DNA, 19 bp, Artificial Sequence
  Synthetic
gcgattccttcactgatac

SEQ ID NO: 21 - DNA, 18 bp, Artificial Sequence
  Synthetic
gcattatgagcatcccca

SEQ ID NO: 22 - DNA, 20 bp, Artificial Sequence
  Synthetic
caaataatgtgattagagat

SEQ ID NO: 23 - DNA, 20 bp, Artificial Sequence
  Synthetic
ttacagggaaaatattgcca

SEQ ID NO: 24 - DNA, 89 bp, Homo sapiens
gaattttacaacaataaaaaatgcaagtggatattgaacagtctctgctctgataattctaaatacagtacagttcacgcccctcacaa

(19) United States
(12) Patent Application Publication
(10) Pub. No.: US 2008/0248995 A1
(43) Pub. Date: Oct. 9, 2008

(54) Modulation of PPARgamma2 gene promoter by FOXO1

(75) Inventors: Eddy Karnieli, Kiryat-Tivon (IL); Michal Armoni, Haifa (IL)

Correspondence Address: BROWDY AND NEIMARK, P.L.L.C.;624 NINTH STREET, NW, SUITE 300, WASHINGTON, DC, 20001-5303, US

(73) Assignee: Technion Research and Development Foundation Ltd., Haifa (IL)

(21) Appl. No.: 11/806,439

(22) Filed: May 31, 2007

Related U.S. Application Data
(60) Provisional application No. 60/809,346, filed on May 31, 2006.

(51) Int. Cl. A61K 38/02; G01N 33/53; C07K 2/00; A61P 29/00; A61P 25/16; A61P 25/28; A61P 9/00; A61P 3/00

(52) U.S. Cl. 514/2; 435/7.2; 435/7.21; 530/300

(57) ABSTRACT

A method for detecting a modulator of transcription of a human PPAR gene promoter is provided, comprising contacting a candidate compound with a cell transfected with an expression vector containing a heterologous gene operably linked to a PPAR promoter and an additional expression vector containing the FOXO1 gene, and comparing the level of expression of said heterologous gene in the presence of the compound and in the absence thereof, whereby a modulator of transcription of the human PPAR gene promoter is identified. The PPAR gene promoter is preferably the PPARγ2 promoter, and the DNA-binding domain of the FOXO1 protein binds to a sequence encompassing the 63 to 323 bp region of the human PPARγ2 promoter, preferably the 270 to 310 bp region.

### FIELD OF THE INVENTION

The present invention relates to FOXO1-mediated PPARγ gene promoter regulation and to a method for detecting modulators of such regulation. Since FOXO1 and PPRγ are important transcription factors regulating glucose metabolism and insulin responsiveness in insulin-target tissues, these modulators are, inter alia, candidates for prevention or treatment of insulin resistance, diabetes and obesity.

Abbreviations: BSA, bovine serum albumin; ChIP, chromatin immunoprecipitation; DBD, DNA binding domain; EMSA, electromobility shift assays; FKHR, forkhead homologue rhabdomyosarcoma; FOXO1, forkhead box o1; IRE or IRS, insulin response element or insulin response sequence; PGC, PPARγ coactivator; PPAR, peroxisome proliferator-activated receptor; PPRγ, peroxisome proliferator-activated receptor-gamma; PPRE, PPAR response element; PRA—primary rat adipocytes; RT-PCR, reverse transcriptase polymerase chain reaction; SDS-PAGE, sodium dodecyl sulfate-polyacrylamide gel electrophoresis; TBE, Tris-borate-EDTA buffer.

### BACKGROUND OF THE INVENTION

The peroxisome proliferator-activated receptor (PPAR¹) family of nuclear receptors and the FOXO family of winged-helix/forkhead box factors are two key families of transcription factors that are important players in the regulation of glucose metabolism, insulin secretion, hepatic glucose production and insulin responsiveness. Members of both families are crucial for a multitude of biological processes, including cell cycle, cell death, differentiation, and metabolism, and have prominent roles in insulin signaling pathways. A convergence of nuclear receptors and forkhead pathways in general, and of FOXO1 and PPARγ in particular, have been implicated in the pathophysiological states of insulin resistance and diabetes, supporting the importance of these transcription factors (Arden, K. C., 2004; Tran et al., 2003). However, in spite of their importance to glucose homeostasis and adipocyte differentiation, the molecular mechanism(s) regulating transcription of the PPARγ gene and the roles of both PPARγ and FOXO1 transcription factors in these processes are not fully known.

The PPAR family of ligand-activated transcription factors includes three PPAR isoforms (α, β/δ, γ) that differ in their tissue distribution and ligand specificity. PPARβ/δ is ubiquitously expressed in many tissues; PPARα is predominantly found in hepatocytes, cardiomyocytes, and enterocytes; and PPARγ is mainly expressed in insulin-responsive tissues, where it has a pivotal role in adipocyte differentiation and the expression of adipose-specific genes (Gilde, A. J., and Van Bilsen, M., 2003). There are two PPARγ isotypes, γ1, γ2, which arise from the use of different promoters and alternative splicing (Fajas et al., 1997). PPARγ2 is nearly adipose-specific, while both are expressed in muscle. We have shown that in primary adipocytes both PPARγ1 and PPARγ2 repress GLUT4 transcription via direct and specific binding of the heterodimer PPARγ/RXRα to a GLUT4 promoter region (Armoni, 2003). We discovered that rosiglitazone—an important thiazolidinedione ligand of PPARγ that improves insulin sensitivity—exerts its beneficial effect on insulin action by detaching PPARγ from its binding site on the GLUT4 promoter, thus alleviating this trans-repression. However, the mechanisms regulating the PPARγ gene promoter itself are largely unknown.

The winged-helix/forkhead family of transcription factors is characterized by a 100-amino-acid, monomeric DNA-binding domain called Forkhead Box (FOX). The DNA-binding domain folds into a variant of the helix-turn-helix motif and is made up of three helices and two characteristic large loops, or “wings”, hence the DNA-binding motif has been named the winged helix DNA-binding domain. Other portions of the forkhead proteins, such as the DNA transactivation or DNA transrepression domains, are highly divergent (Kaestner et al., 2000). The forkhead domain is responsible for DNA binding specificity and binds DNA as a monomer. Following a standardized nomenclature for these proteins (Kaestner et al., 2000), all uppercase letters are used for human (e.g., FOXO1), and only the first letter capitalized for mouse (e.g., Foxo1). The FOXO family of transcription factors stimulates the transcription of target genes involved in many fundamental cell processes, including cell survival, cell cycle progression, DNA repair, and insulin sensitivity (reviewed in Tran et al., 2003). FOXO1 is the most abundant FOXO isoform in insulin-responsive tissues such as hepatic, adipose, and pancreatic cells. Studies show that FOXO1 is negatively regulated by the human PKB/Akt, a serine/threonine kinase that lies downstream of PI3 kinase in the insulin signaling cascade, and that this regulation includes a rapid and hierarchic phosphorylation of FOXO1 on three PKB/Akt phosphorylation consensus sites, T24, S256, and S319 (Tran et al., 2003). Nakae et al (2002) showed that the murine Foxo1 is mainly expressed in adipose tissue and is a negative regulator of insulin sensitivity in liver, pancreatic β-cells, and adipocytes. Impaired insulin signalling to Foxo1 provides a unifying mechanism for the metabolic abnormalities of type 2 diabetes. Studying the importance of FOXO1 to both insulin signaling and tumorigenesis, we have shown that FOXO1 (previously FKHR) either represses or activates transcription from the GLUT4 gene, depending on the cell type, while PAIRED BOX GENE 3/FKHR (PAX3/FKHR), a chimeric gene product that is unique to human alveolar rhabdomyosarcoma, enhances GLUT4 promoter activity via direct binding to specific promoter regions (Armoni et al., 2002).

Although both PPARγ and FOXO1 are main transcription factors in adipose tissue, that are both involved in adipogenesis and insulin signalling, their interactions have rarely been evaluated in bona fide insulin target cells. Furthermore, although PPARγ is involved in multiple regulatory processes, the mechanisms regulating transcription of the PPARγ gene itself are still unknown.

### SUMMARY OF THE INVENTION

1.The present invention provides a method for detecting a modulator of transcription of a human PPAR gene promoter, comprising contacting a candidate compound with a cell transfected with an expression vector containing a heterologous gene operably linked to a PPAR promoter and an additional expression vector containing the FOXO1 gene encoding the FOXO1 protein, and comparing the level of expression of said heterologous gene in the presence of the compound and in the absence thereof, whereby a modulator of transcription of the human PPAR gene promoter is identified.

The PPAR gene promoter may be the promoter of any of the PPAR isotypes and is preferably a PPARγ gene promoter, more specifically the PPARγ2 or PPARγ1 gene promoter.

The present invention also provides compounds detectable by the above method and pharmaceutical compositions comprising them.

### BRIEF DESCRIPTION OF THE DRAWINGS

FIGS. 1A-1C depict gene expression in primary rat adipocytes. FIG. 1A: Endogenous Expression of mRNA by RT-PCR analysis. Total RNA was isolated from primary rat adipocytes and subjected to RT-PCR analysis, as described in the “Experimental Procedures”. Specific primers corresponding to β-actin, GLUT4, FOXO1, total PPARγ and PPARγ2 were used to amplify these mRNA species, as indicated. Aliquots of PCR products (10 μl) were separated by electrophoresis in 2% agarose gels and then stained with ethidium bromide. All PCR products were of the expected size, based on molecular weight markers (MW) shown in the left lane of each panel. Samples from experiments performed in the absence of reverse transcriptase, to exclude the possibility of amplification from genomic contamination, are also shown (no RT). FIGS. 1B-1C: Western blot analysis of FOXO1. FOXO1 immunoreactivity was assessed in total cell lysate, cytosol and nuclear extract fractions of non transfected cells (FIG. 1B) or in total cell lysate prepared from mock-transfected and FOXO1-transfected cells (FIG. 1C), prepared as described in the “Experimental Procedures”. Samples containing 10 μg of protein were subjected to Western analysis. Immunoreactivity was visualized by ECL using anti-FOXO1 as primary antibody, and the relevant bands are shown. The size of FOXO1 protein bands was estimated as approximately 70 kDa on the basis of molecular weight markers (not shown).

FIGS. 2A-2B show dose-dependent effects of FOXO1 and insulin on PPARγ1 and PPARγ2 promoter activities. Isolated adipocytes were co-transfected with 2 μg DNA of the human PPARγ1 (FIG. 2A) or the human PPARγ2 (FIG. 2B) promoter reporters, together with 0 to 5 μg of wild type pcDNA3-FLAG-FOXO1. The empty vector was added to keep the total amount of DNA transfected constant. Cells were incubated in serum-free medium supplemented with 3.5% BSA and either without (basal) or with 100 nM insulin (insulin), and were grown for 20-24 hrs until harvesting. PPARγ promoter activity was determined by measuring luciferase and β-galactosidase activities, as described in the “Experimental Procedures”. Within each experiment, the results are expressed as a percentage of the basal PPARγ promoter activity, i.e., the activity obtained when each promoter reporter was expressed alone. The data are expressed as mean ±SEM of 4 experiments, with each sample analyzed in quadruplicate.

FIGS. 3A-3D show differential contribution of FOXO1 domains to PPARγ1 and PPARγ2 promoter activities. FIG. 3A: Schematic representation of wild type and mutant FOXO1. A series of FLAG-tagged FOXO1 mutants in each of which a different functional domain was incapacitated by point mutation that generates proteins that are defective in either the DBD or at one or all of the three PKB/Akt phosphoacceptor sites, was used; the mutated amino acids are indicated and numbered. NLS, nuclear localization sequence; NES, nuclear export sequence. Isolated adipocytes were transfected with either the human PPARγ1 (FIG. 3B) or the human PPARγ2 (FIG. 3C) promoter reporters, together with 5 μg of wild type or mutated pcDNA3-FLAG-FOXO1. Cells were incubated in serum-free medium supplemented with 3.5% BSA and either without (basal, white bars) or with 100 nM insulin (insulin, black bars), and were grown for 20-24 hrs until harvesting. PPARγ promoter activity was determined by measuring luciferase and β-galactosidase activities, and the data are expressed as mean ±SEM of 3-6 experiments, with each sample analyzed in quadruplicates. FIG. 3D: Exogenous expression of FLAG-tagged FOXO1 proteins. Isolated adipocytes transfected with either the wild type or mutated FLAG-tagged FOXO1 were treated exactly as above. The next day, total cell lysate was prepared from the cells and samples of 10 μg protein were subjected to Western blot analysis. The size of FOXO1 protein bands was estimated as approximately 70 kDa on the basis of molecular weight markers (data not shown). Results of one representative blot (out of three) are presented in the top panel, and data of the quantitative analysis are presented in the histograms below.

FIG. 4 depicts differential contribution of FOXO1 domains to the subcellular localization of FOXO1 protein. HEK-293 cells were transfected with wild type or mutated pcDNA3-FLAG-FOXO1. After 24 hrs, cells were transferred to serum-free medium supplemented with 3.5% BSA and either without (basal) or with 100 mM insulin (insulin), and incubated for 20-24 hrs before staining. At that time, the cells were fixed, permeabilized, and subjected to indirect immunofluorescence staining using an anti-FLAG primary antibody followed by a Cy3-conjugated goat anti-mouse secondary antibody. Nuclei were stained with DAPI. Combined images (DAPI-Cy3) were generated using an Olympus IX81 inverted fluorescent microscope and digital camera, with a DP controller and DP manager software. In the wild type, FOXO1 can be seen all over the cell, mostly in the nuclei in the basal condition, but it is excluded from the nuclei after insulin incubation. This exclusion is prevented in most of the mutants, as can be seen from the clearer blue (DAPI) nuclear staining.

FIGS. 5A-5B show progressive 5′-deletion analysis of the human PPARγ2 promoter. FIG. 5A shows PPARγ2 promoter reporters. The full-length hPPARγ2 promoter reporter and a series of progressive 5′-deletion mutants were generated as detailed in the “Experimental Procedures”. The deletion points used to generate each construct are indicated and numbered. FIG. 5B shows hPPARγ2 promoter activity in primary rat adipocytes. Cells were transiently co-transfected with 2 μg of the various promoter-reporter constructs, as indicated, along with 5 μg of either the pcDNA3 expression vector alone (white bars) or pcDNA3-FOXO1 (black bars). PPARγ2 promoter activity was determined by measuring luciferase and β-galactosidase activities, as described in the “Experimental Procedures”. The data are expressed as mean ±SEM of 4 experiments, performed in quadruplicates.

FIGS. 6A-6B show FOXO1 binding to hPPARγ2 promoter in vitro; Electromobility Shift Assay (EMSA). FIG. 6A: In vitro translation of FOXO1. The integrity and correct size of the in vitro translated proteins for use in EMSA studies were confirmed in parallel reactions performed in the presence of ³⁵S-methionine. The resulting translation products were subjected to 10% SDS-PAGE, followed by phosphor imager analysis. The size of the band is indicated. FIG. 6B: EMSA and supershift. Binding reactions for EMSA included the ³²P-labeled synthetic oligonucleotide, representing region 270-310 on hPPARγ2, and FLAG-tagged FOXO1 protein lysate translated in vitro, as indicated above each lane. An unlabeled bp 270-310 oligonucleotide was used as a specific DNA competitor, and the fold molar excess of competitor is indicated above the relevant lanes. The complex was super shifted by addition of either anti-FLAG or anti-FOXO1 antibodies, as indicated, 10 min prior to addition of the probe. Black and white arrows indicate positions of the bound and free probes, respectively. The dotted arrows indicate the supershift in the presence of the indicated antibodies.

FIGS. 7A-7B show FOXO1 binding to hPPARγ2 promoter in vivo: Chromatin Immunoprecipitation (ChIP). Results represent ChIP assays performed in HEK-293 cells that were transfected with FOXO1 in pcDNA3-FLAG expression vector and with either pGL2-LUC alone or pGL2-LUC-3×IRS promoter reporter. 48 hrs post transfection, DNA and protein were subjected to either one-step (FIG. 7A) or two-step cross-linking (FIG. 7B) (Nowak et al., 2005). Cells were lyzed and sonicated as explained in “Experimental Procedures”. An aliquot of whole cell lysate was removed for purification of total DNA (T), and immunoprecipitations were conducted using either anti-FOXO1 antibody (αFOXO1) or negative control IgG (αIgG). DNA was extracted from the immunoprecipitates, and PCR (26 cycles) was conducted on total DNA and immunoprecipitated DNA with primers corresponding to promoter region 63-323 of the human PPARγ2 gene (FOXO1-hPPARγ2), or to region encompassing the 3×IRS sequence in pGL2 (3×IRS). PCR products were analyzed on 2% agarose gel, and visualized by ethidium bromide staining, in presence of DNA molecular weight markers (MW). Data from one representative assay (out of three) are shown.

FIG. 8 shows suggested model for FOXO1 regulation of PPARγ1 and PPARγ2 promoters. Based on the data we obtained from 5-del and EMSA analyses on one hand, and FOXO1 mutation analysis on the other, we suggest the following model for FOXO1 repression of PPARγ1 and PPARγ2 promoters. 1) As shown in FIG. 1 in the basal state FOXO1 recycles between the nucleus and the cytoplasm, and is mostly localized to the nucleus. 2) Upon insulin stimulation, insulin signaling proceeds to PKB/Akt; 3) PKB/Akt activation leads to hierarchic phosphorylation of FOXO1 on three PKB/Akt consensus sites (depicted as circled P), T24, S256 and S319. 4) PKB/Akt phosphorylation of FOXO1 on either of these sites leads to its nuclear exclusion, followed by either complete or partial derepression of PPARγ1 or PPARγ2 promoter activities, respectively. 5) Once in the nucleus, FOXO1 binds directly to the PPARγ2 promoter, via at least one specific DNA sequence encompassing 270-310 bp. This leads to a dose-dependent repression of PPARγ2 transcriptional activity (represented by bold down-arrows). Mutations in either one of the PKB/Akt phosphorylation site, or in the FOXO1 DNA binding domain, that render FOXO1 protein either refractory to PKB/Akt, or defective in binding ability, respectively, lead to partial derepression of PPARγ2 promoter. 6) FOXO1 also represses transcription from the PPARγ1 promoter, however, this effect probably does not include direct binding to the PPARγ1 promoter, as a H215R mutant that has defective binding capacity, still represses the promoter. Thus, repression of PPARγ1 promoter by FOXO1 probably occurs via a different pathway than that of PPARγ2, and involves an indirect regulation via a mediator, the nature of which warrants further investigation.

FIG. 9 depicts a paradigm for FOXO1 effects to increase insulin sensitivity in adipocytes. Based on our findings from this and previous studies, we suggest the following paradigm for FOXO1 enhancement of insulin sensitivity: 1) FOXO1 represses gene expression of PPARγ1 and PPARγ2, either indirectly or directly, respectively; 2) PPARγ1 and PPARγ2 repress gene expression of GLUT4. 3) Thus FOXO1, directly or indirectly leads to derepression and/or activation of GLUT4, which 4) subsequently results in enhanced insulin sensitivity. Flat-headed arrows denote gene repression and pointed-head arrows denote activation.

### DETAILED DESCRIPTION OF THE INVENTION

Failure to respond to insulin is a prominent feature in insulin resistance, type 2 diabetes, and obesity. It has now been found, in accordance with the present invention, that the direct and specific interaction between FOXO1 (SEQ ID NO: 1) and the promoter of PPARγ2 (SEQ ID NO: 2) represses the transcription of the PPARγ2 gene and that this, in turn, up-regulates the expression of the GLUT-4 gene and increases responsiveness of the cell to insulin.

It has also been found in accordance with the present invention that the DNA region within the PPARγ2 promoter to which FOXO1 binds directly and specifically and which mediates FOXO1 effects on PPARγ2 is between base pairs 63-323 (SEQ ID NO: 3). In a preferred embodiment, the present invention demonstrates a novel FOXO1 binding motif encompassing bp 270-310 (SEQ ID NO: 4).

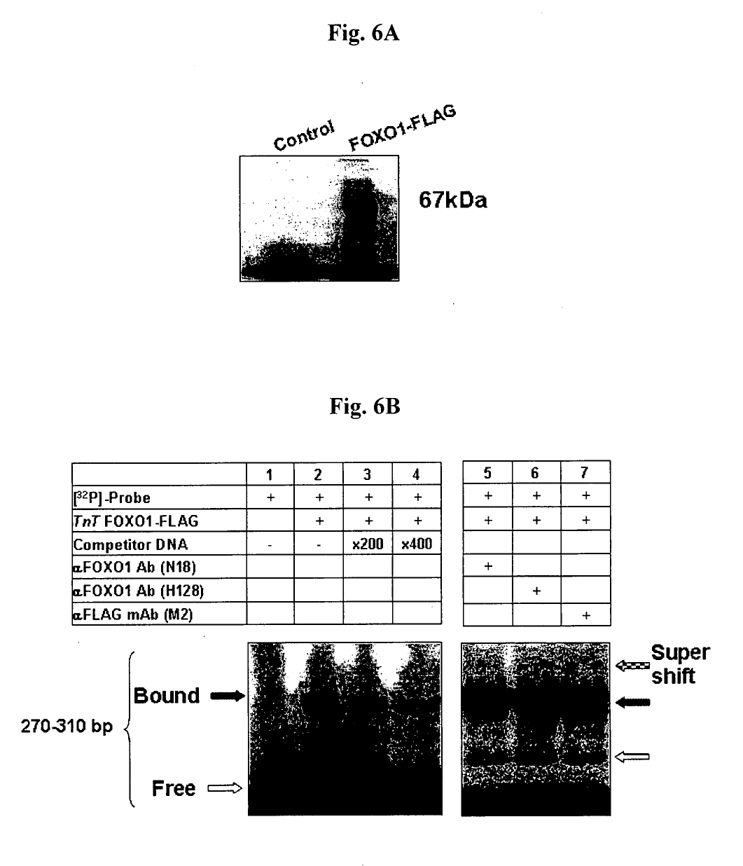

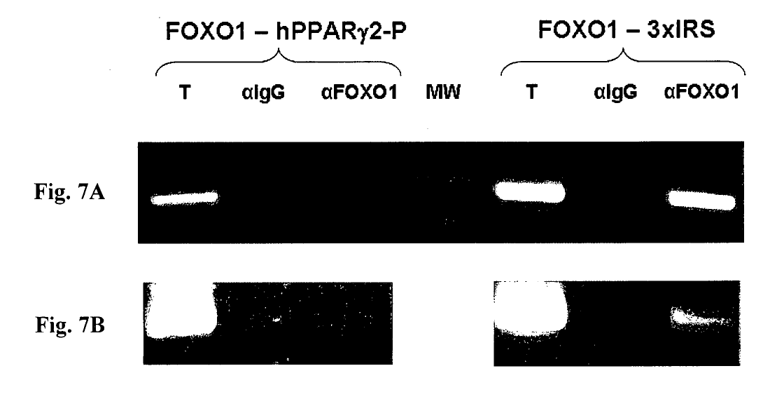

We screened the PPARγ2 promoter (hG2-P587) for the presence of cis-elements that may serve as potential FOXO1 binding sites. Binding-site selection studies performed with a variety of forkhead proteins have led to the identification of a core recognition motif, T-(G/A)-T-T-(G/T)-(G/A)-(C/T) (SEQ ID NO: 5), that is necessary for forkhead binding, while bases immediately flanking this core contribute to the binding specificity of the different family members; for example, the optimal DNA-binding site for the FOXO members has been determined to be TTGTTTAC (SEQ ID NO: 6) (Burgering and Kops, 2002). We have found that this core recognition motif is present at position 431 bp of hG2-P587. However, as evident from the 5′-deletion and ChIP analyses we preformed, this motif lies beyond the region which was found to mediate most of FOXO1 effects on PPARγ2 and bound it in a direct and specific manner (FIGS. 6A and 6B and FIGS. 7A and 7B). Thus, we show for the first time that, while PPARγ1 may be indirectly regulated by FOXO1, regulation of transcriptional activity from the hPPARγ2 promoter occurs via direct and specific binding of FOXO1 to a novel yet unidentified response element on PPARγ2-P. This FOXO1 response element/binding motif lies in a region that encompasses bp 270-310 of PPARγ2-P, and probably extends to further upstream and downstream regions, as evident from our ChIP and 5-deletion analyses.

Further screening of the 270-310 bp region for known motif sequences revealed that it contains response elements for PPARγ and E2F, two factors that were found to regulate PPARγ transcription. The E2F response element is similar to that found by Fajas et al on the PPARγ1 promoter, and is associated with induction of PPARγ1 transcription during clonal expansion of 3T3-L1 adipocytes (Fajas et al., 2002). Interestingly, the PPARγ response element (PPRE) we found is similar to the acyl-coenzyme A oxidase PPRE included in the (AOX)₃-Luc reporter. This PPRE may contribute to FOXO1 regulation of the PPARγ promoter via a complex mechanism that involves a FOXO1-PPARγ interaction. Indeed, Dowell et al (2003) have shown that Foxo1 and PPARγ functionally interact in a reciprocally antagonistic manner. Consistent with this, our data support the notion of a convergence of PPARγ and FOXO1 signaling in the action of insulin. A more general convergence of nuclear receptors and forkhead factor pathways may be important for multiple biological processes, and this convergence may be evolutionarily conserved.

The present invention is based on the finding by the inventors that the human winged/helix transcription factor FOXO1 represses transcription from the human PPARγ1 (SEQ ID NO: 7) and PPARγ2 gene promoters in bona fide insulin target cells, and that this, in turn, up-regulates the expression of the GLUT-4 gene and increases responsiveness of the cell to insulin. This regulation is both insulin-dependent and isoform-specific.

1.The invention thus provides a method for detecting a modulator of transcription of a human PPAR gene promoter, comprising contacting a candidate compound with a cell transfected with an expression vector containing a heterologous gene operably linked to a PPAR promoter and an additional expression vector containing the FOXO1 gene encoding the FOXO1 protein, and comparing the level of expression of said heterologous gene in the presence of the compound and in the absence thereof, whereby a modulator of transcription of the human PPAR gene promoter is identified.

Although FOXO1 is endogenously expressed in insulin responsive cells, it is advantageous to co-transfect the cells with a vector expressing FOXO1 in addition to the reporter gene vector, because its exogenous expression is about 20-fold higher than the endogenous expression.

2.The PPAR gene promoter may be derived from any of the PPAR isotypes, namely, PPARα (SEQ ID NOs: 8-11), PPARβ\δ (SEQ ID NO: 12) or PPARγ gene promoter. In one preferred embodiment, the PPAR gene promoter is a PPARγ gene promoter, namely, the PPARγ or PPARγ2 gene promoter.

2. A role for FOXO1 is emerging as both transcription activator and repressor of nuclear receptors (Tran et al., 2003). Looking at the protein structure of FOXO1, it is apparent that besides a proline-rich and acidic serine/threonine-rich region that serves as a DNA activation domain at the C terminus, it also contains an alanine-rich region at its N-terminus, which is believed to serve as a potential transcriptional repression domain, and an area in its mid-region thought to mediate the interactions with nuclear receptors (Tran et al., 2003). This suggests that, depending on the specific milieu and cellular distribution, FOXO1 can act as either trans-repressor or trans-activator of PPARγ gene transcription. Interestingly, the inventors have shown that it is the adipose-specific isoform, PPARγ2 that is differentially regulated by FOXO1 in preadipocytes vs adipocytes, being immensely activated in the predifferentiated stage and trans-repressed in the fully differentiated state (unpublished data). These findings are supported by the work of Fajas et al showing that E2F4 triggers the expression of PPARγ in preadipocytes, resulting in differentiation into adipocytes, but when cells are terminally differentiated, E2F4 represses PPARγ gene expression through an association with p130/p107 (Fajas et al., 2002). In all, the data disclosed herein point to a unique role for FOXO1-regulated PPARγ2 expression during adipogenesis, where FOXO1 greatly enhances PPARγ2 expression in pre-differentiated cells while repressing it once PPARγ2 has completed its duties as master regulator of adipogenesis.

In one embodiment, the modulator represses the human PPARγ promoter and reduces the level of expression of the heterologous gene. This means that in vivo the expression of the PPARγ1 or PPARγ2 gene will be repressed.

3.In another embodiment, the modulator activates the human PPARγ promoter and increases the level of expression of the heterologous gene. This means that in vivo the expression of the PPARγ1 or PPARγ2 gene will be activated.

Based on the finding of the present invention that the DNA binding site of FOXO1 protein binds to a sequence of the PPARγ2 gene promoter, in one embodiment of the method of the invention the modulator tested affects the FOXO1 binding to the PPARγ2 gene promoter such as causing repression of the heterologous gene expression directly. This means that such a compound will in vivo repress the expression of the PPARγ2 gene. In another embodiment, the modulator tested affects the FOXO1 binding to the PPARγ2 gene promoter such as causing activation of the heterologous gene expression. This means that such a compound will in vivo activate the expression of the PPARγ2 gene directly.

It was found according to the present invention that the DNA-binding domain of the FOXO1 protein (SEQ ID NO: 13) binds to a sequence encompassing the 81 to 325 bp region, preferably 270-310 region, of the human PPARγ2 promoter, leading to repression of the PPARγ2 gene promoter and consequently of the PPARγ2 gene expression, and that this binding can be demonstrated in cellulo within the context on the human nucleosome. This newly-identified FOXO1 response element may thus be used as a molecular therapeutic target for the treatment of insulin resistance, diabetes type 2 and obesity.

The present invention thus also relates to the use of the DNA sequence of the PPARγ2 gene promoter to which the FOXO1 protein binds, as a molecular therapeutic target for the treatment of insulin resistance, diabetes type 2 and obesity, wherein said DNA sequence is comprised within the 81 to 325 bp region, preferably the 270-310 bp region, of the human PPARγ2 promoter.

9.The findings of the instant invention gain support from a large body of evidence, showing that the effects of FOXO1 we observed in primary adipocytes indeed reflect bona fide features of FOXO1-regulated PPARγ gene expression either in cellulo or in vivo. Firstly, knockout studies by Accili and colleagues have shown that Foxo1 haploinsufficiency is associated with a significant enhancement of PPARγ MRNA levels in epididymal adipocytes, (Nakae et al., 2003). Secondly, arguing that dominant-negative Foxo1 mutants provide a useful reagent to study the effects of Foxo1 knockout in experimental systems, Accili and colleagues showed that transduction of 3T3-L1 adipocyte with Foxo1-A256 lead to earlier induction of adipocyte differentiation which is paralleled by an earlier induction of PPARγ gene expression (Nakae et al., 2003). Thirdly, Kloting et al (Kloting et al., 2006), have shown that in the Wistar Ottawa Karlsburg W (WOKW) rat model of human metabolic syndrome, severe insulin resistance is associated with increased Foxo1 levels in epididymal adipocytes, in accordance with decreased PPARγ gene expression. Fourth, using promoter-reporter studies, Dowell et al (2003) have shown that Foxo1 and PPARγ functionally interact in a reciprocally antagonistic manner. All these findings support the findings introduced herein, that the effects of FOXO1 observed reflect genuine features of PPARγ gene expression.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

In one embodiment, the heterologous gene is a reporter gene. Non-limiting examples of heterologous genes include reporter genes such as luciferase, chloramphenicol acetyl transferase, beta-galactosidase, and the like which can be juxtaposed or joined to heterologous control regions or to heterologous polypeptides.

The findings disclosed herein underscore the importance of studying the regulation of the PPARγ gene expression in context of genuine insulin target cells, as in the present study. Adipogenesis is regulated by the hormonally induced coordinated expression and activation of two main groups of transcription factors, the CCAAT/enhancer binding protein (C/EBP) family and PPARγ (Spiegelman, and Flier, 1996). Due to its pivotal role in adipocyte differentiation and in expression of adipocyte-specific genes, the PPARγ receptor is often called “master of adipogenesis”. The adipogenic transcription factors then induce the expression of adipocyte-specific genes, ultimately leading to a morphologically distinct and functional fat cell.

The cells used in method of the invention are preferably insulin-responsive cell such as but not limited to, adipocytes, smooth muscle cells, skeletal muscle cells or cardiac muscle cells. In one preferred embodiment, the cells are adipocytes. The cells may also be non-insulin responsive cells such as brain, liver, gut or pancreas cell.

In another aspect, the present invention relates to a compound capable of modulating the FOXO1-mediated PPARγ1 or PPARγ2 gene expression. In one embodiment, the compound represses FOXO1-mediated PPARγ1 or PPARγ2 gene expression directly or indirectly. In another embodiment, the compound activates FOXO1-mediated PPARγ1 or PPARγ2 gene expression directly or indirectly. The invention encompasses such compounds detectable by the method of the invention or by any other method.

The compound of the invention may be a small organic molecule identified from a chemical library or it may be a peptide identified from a peptide library. In one embodiment, the peptide is derived from the FOXO1 DNA-binding domain or an analog of said peptide.

In a further aspect, the invention provides a pharmaceutical composition for treatment, attenuation, or prevention of insulin resistance, type 2 diabetes and obesity, comprising a pharmaceutically acceptable carrier and a compound of the invention that represses FOXO1-mediated PPARγ1 or PPARγ2 gene promoter and, consequently, represses PPARγ1 or PPARγ2 gene expression.

In another aspect, the invention relates to a pharmaceutical composition for treatment, attenuation, or prevention of a human disease or disorder that is affected by activation of a PPARγ gene expression, comprising a pharmaceutically acceptable carrier and a compound of the invention that activates FOXO1-mediated PPARγ1 or PPARγ2 gene promoter and, consequently, activates PPARγ1 or PPARγ2 gene expression. Non-limiting examples of such diseases or disorders include atherosclerosis, coronary events, brain inflammation, and a neurodegenerative disease such as multiple sclerosis, Parkinson's disease and Alzheimer's disease.

It is shown herein in the examples that the effects of FOXO1 observed in primary adipocytes indeed reflect bona fide features of FOXO1-regulated PPARγ gene expression either in cellulo or in vivo. Furthermore, GLUT4 gene expression directly correlates to FOXO1 levels in bona fide insulin target cells (Armoni et al., 2002; Armoni et al., 2006). Therefore, the cell used is preferably an insulin responsive cell, and most preferably an adipocyte. In a preferred embodiment, the heterologous gene is a reporter gene.

The reporter gene used in the invention can be chosen from any one of those commonly used in the art, for example, but not limited to, the gene that encodes jellyfish green fluorescent protein, which causes cells that express it to glow green under UV light. Another reporter gene codes for the enzyme luciferase, which catalyzes a reaction with luciferin to produce light. Other reporters commonly used are GUS (beta-glucuronidase), which creates blue coloration when transformed cells or tissues are provided with the appropriate substrate, chloramphenicol acetyltransferase (CAT) that neutralizes chloramphenicol, and bacterial nitroreductase (NTR) that uses a cell permeant cyanine fluor, CytoCy5S, as its substrate. In a preferred embodiment of the instant invention luciferase is used as reporter gene.

According to the method of the invention, the transfected cells are contacted with candidate compounds that may affect the transcription of the PPARγ2 gene. The compounds may be small organic compounds or peptides. Standard methods for measuring the activity of the reporter gene are then used in the presence or in the absence of the compounds, and compounds effective in modulating the PPARγ2 gene transcription are identified.

Insulin and other growth factors are known to promote phosphorylation of FOXO1 and PPARγ on phosphoacceptor sites, resulting in changes in the intracellular localization and activity of these transcription factors. The transcriptional activity of FOXO1 is regulated by insulin at the levels of transactivation, DNA binding, and nuclear exclusion. These different regulatory mechanisms allow the precise control of transcription of FOXO1 target genes by insulin. In one embodiment of this invention it is shown that the insulin effects on the subcellular distribution of FOXO1 can be segregated from its effect on the transcriptional activity of PPARγ. It was shown that a mutant with defective DNA binding, H215R, retained its ability to repress PPARγ1 while losing its regulatory effects on the PPARγ2 promoter. These findings show that an intact DNA binding domain (DBD) is crucial for FOXO1 regulation of the PPARγ2 (but not PPARγ1) promoter, and suggest the involvement of direct binding of FOXO1 to PPARγ2 promoter. As the DBD region of FOXO1 is not necessary for transcriptional repression of the PPARγ1 isoform; however, FOXO1 likely has an indirect effect on this isoform, perhaps via some mediating protein(s). Indeed, Zhao et al (2001) found that FOXO1 can interact with both steroid and non-steroid nuclear receptors in either a ligand-dependent or independent manner to differentially regulate the transactivation mediated by different nuclear receptors. This identifies FOXO1 as a bifunctional transcription factor that functions as both a co-activator and a co-repressor of the PPARγ promoter, via either direct or indirect interactions. In addition to the H215 phosphorylation site present in the DBD, there are three key regulatory residues that are conserved within the FOXO family. Analysis of the differential contribution of these FOXO1 PKB/Akt phosphoacceptor sites demonstrated that while a T24A mutation in FOXO1 affects neither its basal nor its insulin-mediated capacity to repress the PPARγ1 promoter in primary adipocytes, it is associated with major loss of FOXO1's ability to repress the PPARγ2 promoter in the basal state. Thus, it is apparent that phosphorylation of FOXO1 in general, and of the T24 site in particular, contributes to FOXO1-mediated transcriptional activity in a manner that is both tissue- and species-specific. Interestingly, there are data pointing to a unique role for FOXO1-regulated PPARγ2 expression during adipogenesis, where FOXO1 greatly enhances PPARγ2 expression in pre-differentiated cells while repressing it once PPARγ2 has completed its duties as master regulator of adipogenesis.

There are three closely related homologous of PPAR: The expression of PPARα is highest in liver, kidney and heart, while the expression of PPARβ/δ is highest in adipose tissue, skin and brain, but is widespread in many tissues. Expression of PPARγ receptor is highest in adipose tissue, but it is also highly expressed in other cell types like macrophages (Semple et al., 2006). PPARγ is involved in the regulation of macrophage differentiation and activation in the peripheral organs, and PPARγ natural and synthetic agonists may control brain inflammation by inhibiting several functions associated to microglial activation, such as the expression of surface antigens and the synthesis of nitric oxide, prostaglandins, inflammatory cytokines and chemokines. In addition to microglia, PPARγ agonists affect functions and survival of other neural cells, including astrocytes, oligodendrocytes and neurons. PPARγ activators may provide protection against atherosclerosis and coronary events (Kurtz, 2006; Li and Palinski, 2006). Although most of the evidence comes from in vitro observations, an increasing number of studies in animal models further supports the potential therapeutic use of PPARγ agonists in human brain diseases including multiple sclerosis, Parkinson's disease and Alzheimer's disease. (Bernardo and Minghetti, 2006). Therefore, depending on the specific milieu and cellular distribution, FOXO1 can act as either trans-repressor or trans-activator of PPARγ gene transcription.

An additional aspect of the invention relates to the identification of peptides that can modulate the transcription of the PPARγ2 gene similarly to the native whole FOXO1 protein. The amino acid sequence of these peptides, and thus their tertiary structure, can be derived from the DNA binding domain of FOXO1. Alternatively, analogs of these peptides can differ in their primary structure from the DNA binding domain of FOXO1, but still be homologues with it regarding their tertiary structure.

In summary, FOXO1 and PPARγ are crucial transcription factors regulating glucose metabolism and insulin responsiveness in insulin target tissues. We showed that in primary rat adipocytes (PRA) both factors regulate transcription of the insulin-responsive GLUT4 gene, and that PPARγ2 detachment from the GLUT4 promoter upon thiazolidinediones binding upregulates GLUT4 gene expression, thus increasing insulin sensitivity (Armoni et al., 2003). However, the mechanisms regulating PPARγ gene transcription are largely unknown. We studied the effects of FOXO1 on human PPARγ gene expression in PRA, where we found that both genes are endogenously expressed. FOXO1 co-expression dose-dependently repressed transcription from either the PPARγ1-P or the PPARγ2-P reporters by 65%, while insulin (100 nM, 20-24 hrs) either partially or completely reversed this effect. Phosphorylation-defective FOXO1 mutants T24A, S256A, S319A and AAA still repressed PPARγ1-P, while partially loosing their effects on PPARγ2-P in either basal or insulin-stimulated cells. Using a DNA binding-defective FOXO1 (H215R) indicated that this domain is crucial for FOXO1 repression of PPARγ2-P, but not PPARγ1-P. Progressive 5′-deletion and gel retardation analyses revealed that this repression involves a direct and specific binding to FOXO1 to PPARγ2-P; ChIP analysis confirmed that this binding occurs in cellulo. We suggest a novel paradigm to increase insulin sensitivity in adipocytes, where FOXO1 repression of PPARγ, the latter being a repressor of GLUT4-P, consequently leads to GLUT4 derepression/upregulation, thus enhancing cellular insulin sensitivity.

The newly-identified FOXO1 binding site on PPARγ2-P may serve as molecular therapeutic target for the development of agents affecting the expression of PPARγ2 that can serve as drug candidates for the treatment of insulin resistance, diabetes type 2 and obesity.

The following examples illustrate certain features of the present invention but are not intended to limit the scope of the present invention.

### EXAMPLES

### Experimental Procedures

(i) Reverse Transcriptase (RT)-PCR—Total cellular RNA was prepared from the various cells using a TriReagent kit (Molecular Research Center, inc., Cincinnati, Ohio), and further purified using RNeasy® columns (Qiagen, GmbH, Germany). Sense and antisense primers specific for β-actin (SEQ ID NOs: 14, 15), FOXO1 (SEQ ID NOs: 16, 17), total PPARγ (SEQ ID NOs: 18, 19), and PPARγ2 (SEQ ID NOs: 20, 21) mRNA were synthesized based on sequences obtained from GenBank. First-strand cDNA synthesis and PCR amplification were performed using a Reverse-iT™ 1^{st} Strand Synthesis kit (AB Gene, Surrey, UK). Experiments performed in the absence of reverse transcriptase (no RT) excluded the possibility of amplification from genomic contamination. PCR products were separated on 2% agarose gels and visualized by ethidium bromide staining, as previously described (Armoni et al., 2002).

(ii) Expression Vectors and Luciferase Promoter Reporters—Expression vectors encoding FOXO1 in pcDNA3 were kindly provided by Dr. Eric Tang (University of Michigan Medical School, Ann Arbor, Mich.) and have been described previously (Tang et al., 1999). These included a construct encoding for the full-length open reading frame of wild type FOXO1 (SEQ ID NO: 1); the constitutively active phosphorylation-defective mutants T24A, S256A, S319A, and AAA; and a DNA binding-defective mutant, H215R. A control synthetic reporter, 3×IRS-LUC, containing three repeats of an insulin response element (IRE) consensus sequence in pGL2-LUC was also provided by Dr. Eric Tang (Tang et al., 1999). The human PPARγ1 and PPARγ2 promoters in pGL3-LUC (hG1-P3000 and hG2-P587, respectively) were obtained from Dr. Luis Fajas (CNRS INSERM, Louis Pasteur University, Strasbourg, France) and have been described previously (Fajas et al., 1997). A series of progressively 5′-deleted promoter reporters was generated from the hG2-P587 reporter using the QuikChange® XL Site-Directed Mutagenesis Kit (Stratagene, La Jolla, Calif.). All sequences were confirmed by direct sequencing.

(iii) Transient Expression and Promoter Reporter Assays—Isolated adipocytes were prepared from rat epididymal fat pads and transfected according to procedures we described previously (Armoni et al., 2003). Briefly, adipocytes were transfected by electroporation (3 pulses×920 V, 50 uF, GenePulser II, BioRad) with 2.0 μg of either PPARγ1 or PPARγ2 promoter reporter DNA, 0 to 5 μg of expression vectors for FOXO1 (wild type or mutants), and 0.5 μg of pCMV-β-galactosidase. One hour later, an equal volume of incubation medium (supplemented with 7% BSA and either with or without 100 nM insulin) was added to the DNA-containing medium and the cells were incubated for additional 20-24 hrs at 37° C. One set of tubes was transfected with the 3×IRS-LUC promoter reporter to be used as a positive control for FOXO1 transcription activation. In each experiment, the total amount of DNA transfected was held constant by adding the relevant insertless expression vector to account for squelching by the promoter itself. Luciferase activity was assayed at room temperature using a Luciferase Reporter Assay Kit (Promega) and a Lumat LB9501 luminometer (Berthold Systems inc., Nashua, N.H.). Luciferase activity was normalized to β-galactosidase activity as internal control (Sambrook et al., 1989). Within each experiment, values were expressed as a percentage of the induced basal PPARγ promoter activity, i.e., the activity obtained in cells transfected with promoter reporter alone. Cell viability was assessed by trypan blue exclusion. Each experiment was repeated 4-6 times, with each sample analyzed in quadruplicates.

(iv) Assessment of FOXO1 Proteins by Western Immunoblotting—Endogenous expression of FOXO1 proteins, and the levels of the exogenously over-expressed FOXO1 (wild type and mutants) were assessed by Western immunoblotting in either total cell lysates or subcellar fractions of nuclear extracts and cytosols. Nuclear and cytosolic fractions were prepared as detailed by us before (Armoni et al., 2005). For preparation of total cell lysates, parallel samples of mock-transfected and FOXO1-transfected primary rat adipocytes, treated exactly as for luciferase reporter assay in 1× Reporter Lysis Buffer® (Promega, Madison, Wis.), were supplemented with 1% SDS and proteases inhibitors, vortexed and spinned down at 4C×1000 g. The upper fat layer was then removed and the infranatant, comprising the total cell lysate fraction, was collected. Cellular protein levels were assessed with the bicinchoninic acid protein assay kit (Pierce Chemical Co., Rockford, Ill.). Samples of 10 μg protein were analyzed by Western immunoblotting using either anti-FLAG M2 monoclonal antibody (Sigma-Aldrich Israel Ltd., Rehovot, Israel) for detection of exogenously over-expressed FLAG-tagged FOXO1 or anti-FOXO1 rabbit polyclonal antibody (Cell Signalling Technology, inc., Beverly, Mass.) for detection of endogenous FOXO1. Antigen-antibody complexes were detected by ECL, with SuperSignal West Pico Chemiluminescent kit (Pierce Chemical Co., Rockford, Ill.). Dried blots were exposed to X-ray films, scanned, and quantitatively analyzed.

(v) Immunofluorescence Studies—HEK-293 cells were plated on 18-mm glass cover slips in 6-well plates at density 25,000 cells/well, and transfected as detailed by us before (Armoni et al., 2003). Cells transfected with expression vectors for either wild type or various mutants of FLAG-tagged FOXO1 were incubated for 24 hrs at 37° C. Next day, cells were washed with magnesium-containing PBS and transferred to serum deprived medium that was supplemented with 2% BSA and either with or without insulin as detailed above. After 24 hrs, the cells were fixed in 4% para-formaldehyde and stained for indirect immunofluorescence using an anti-FLAG primary antibody, followed by a Cy3-conjugated goat anti-mouse secondary antibody. FLAG-tagged FOXO1 proteins and DAPI-stained nuclei were visualized using an Olympus IX81 inverted fluorescent microscope (Olympus, Melville, N.Y.). Combined DAPI-Cy3 images were generated using a DP70 digital camera supplemented with DP Controller and DP Manager Software (Olympus, Melville, N.Y.).

(vi) In vitro Translation and Electrophoretic Mobility Shift Assay (EMSA)—In vitro translation of FOXO1 proteins and EMSA studies were performed as described (Armoni et al., 2003). The TnT SP6/T7-coupled reticulocyte lysate system (Promega, Madison Wis.) was used to generate in vitro translated FLAG-tagged FOXO1 proteins from the corresponding cDNA, and the resulting protein lysate was used in EMSA. Protein expression was confirmed by SDS-PAGE, followed by phosphorimaging analysis of proteins translated in the presence of ³⁵S-methionine (cell labeling grade; Amersham, Buckinghamshire, UK). In vitro translation reactions generated sufficient protein to use in EMSA studies. Sense and antisense PPARγ promoter-derived oligonucleotides were synthesized that correspond to bp 270-310 of hG2-587 (sense: GACACTGAACATG TGGGT CACCGGCGAGACAGTGTGGCAA) (SEQ ID NO: 4); these were annealed and end-labeled with γ-³²P-ATP (6000 Ci/mmol; Amersham, Buckinghamshire, UK) in the presence of polynucleotide kinase. Protein-DNA binding reactions were assembled in a total volume of 20 μl, which included 4 μl of the in vitro translated FOXO1 lysate, radiolabeled probe (˜75,000 cpm), and 4 μg of poly(dI-dC) in a buffer containing 10 mM HEPES (pH 7.9), 1 mM dithiothreitol, 1 mM EDTA, 4% Ficoll, and 50 mM KCl. Competition experiments were performed in the presence of 100- and 200-fold molar excess of unlabeled probe, which was added 10 min prior to the addition of the radiolabeled probe. For supershift assays we used either anti-FLAG M2 monoclonal antibody (Sigma-Aldrich Israel Ltd., Rehovot, Israel) or anti-FOXO1 polyclonal antibodies (FKHR N-18 and FKHR H-128, Santa Cruz Biotechnology, Santa Cruz, Calif.). Protein lysates were preincubated with antisera for 10 min prior to the addition of the labeled probe. After incubation for 30 min at 25° C., DNA-protein complexes were resolved by electrophoresis on 5% non-denaturing PAGE at 150 V and 4° C. in 0.5×TBE buffer (45 mM Tris pH 8.3, 45 mM borate, and 1.0 mM EDTA). Gels were fixed in 10% acetic acid for 15 min, dried, and analyzed by phosphorimaging.

(vii) Chromatin Immunoprecipitation (ChIP) Assays—ChIP assays were performed following the method of Shang et al (2000) with modifications. Human embryonal kidney (HEK)-293 cells were chosen as a source for human PPARγ2 promoter that resides in the context of the human native nucleosome. Cells were transfected with FOXO1 in pcDNA3-FLAG expression vector along with either 3×IRS-LUC (positive control) or empty promoter reporter, as detailed above. The next day, cells were washed with magnesium-containing PBS and incubated with DMEM/2% BSA for additional 24 hours. The next day, cells were washed with magnesium-containing PBS, and cross-linking of DNA and proteins was preformed using a two-step technique, according to Nowak et al (2005). Cells were then sonicated in cell lysis buffer, to generate DNA fragments of average size of 500 bp. One-tenth of the total cell lysate was used for purification of total genomic DNA, and the rest of the lysate was immunocleared with Protein G sepharose and sheared salmon sperm DNA. Samples were spinned down for 10 min at 10K×g, then immunoprecipitated with either anti-FOXO1 or anti-FLAG antibodies, or with negative control IgG (normal rabbit IgG, SC-2027 Santa Cruz, Calif.), at 4° C. for 18 h. Immunoprecipitates were collected using Salmon Sperm DNA/Protein G agarose. DNA was extracted by phenol-chloroform followed by ethanol precipitation, and PCR was then performed using either total DNA or immunoprecipitated DNA. Primers used for PCR corresponded to sequences within the human PPARγ2 promoter region 63-323 bp (SEQ ID NOs: 22-23), and a region encompassing the 3×IRS sequence in PGL2. PCR products were separated on 2.0% agarose gel and visualized by ethidium bromide staining.

### Example 1

### Endogenous Gene Expression

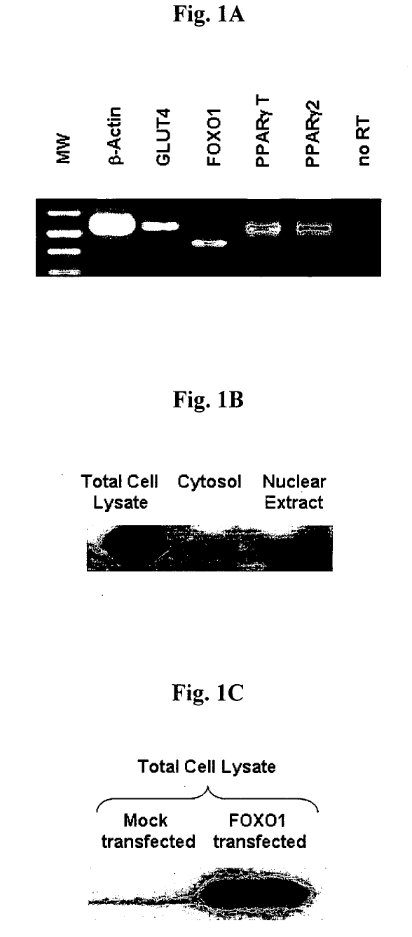

Endogenous gene expression at mRNA and protein level was examined by RT-PCR and Western blot analyses, respectively. As shown in FIG. 1A, isolated primary rat adipocytes showed endogenous expression of mRNA for GLUT4, FOXO1, total PPARγ and PPARγ2. Endogenous expression of GLUT4 was taken as a marker for an insulin-responsive tissue. Western immunoblotting showed endogenous expression of FOXO1 protein in total cell lysates prepared from primary rat adipocytes; under these basal conditions, FOXO1 was localized to the nuclear fraction and undetected in the cytosol (FIG. 1B). We also determined the expression efficiency of FOXO1 in primary rat adipocytes by Western immunoblotting, and found that exogenous FOXO1 is over-expressed to 20-fold of the endogenous protein (FIG. 1C).

### Example 2

### Transcriptional Activity of PPARγ1 and PPARγ2 is Differentially Regulated by FOXO1 and Insulin

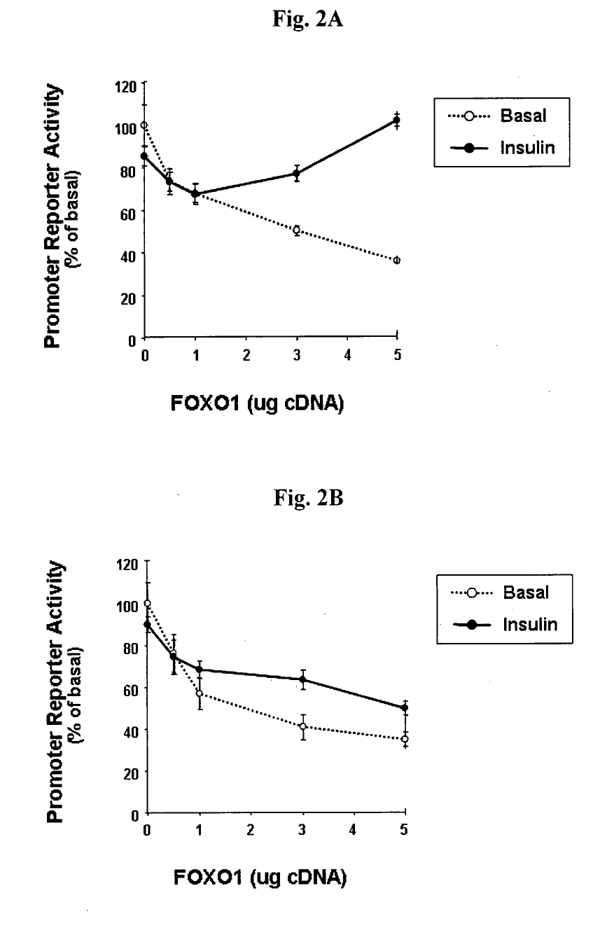

Once establishing the expression patterns of endogenous and exogenous FOXO1 in primary rat adipocytes, we next studied the effects of FOXO1 on human PPARγ1 and PPARγ2 gene expression at transcriptional level. PRA were co-transfected with luciferase-conjugated promoter reporters for either the human PPARγ1 or PPARγ2, (PPARγ1-P and PPARγ2-P, respectively) along with expression vector for wild type FOXO1 (FIGS. 2A-2B). We found that expression of wild type FOXO1 repressed the transcriptional activity of both the co-expressed PPARγ1-P and PPARγ2-P in a dose-dependent manner, to as much as 65% below basal levels. Incubation of cells with 100 nM insulin resulted in a dose-dependent reversal of FOXO1 effects on the PPARγ1 promoter, with transcriptional activity reaching 102±3% of basal level at maximal FOXO1 dose applied. Insulin also interfered with FOXO1 repression of the PPARγ2 promoter, but to a lesser extent. Under similar conditions, FOXO1 activated the IRE from the 3×IRS-LUC reporter, which was used as a positive control, by as much as 8.5-fold (data not shown); this excludes the possibility of either cytotoxic or squelching effects in the expression system. These data show that FOXO1 equally represses transcription from PPARγ1 and PPARγ2′ promoters, while insulin interferes with this effect in an isoform-specific manner.

### Example 3

### Differential Contribution of FOXO1 Domains to PPARγ Promoter Regulation

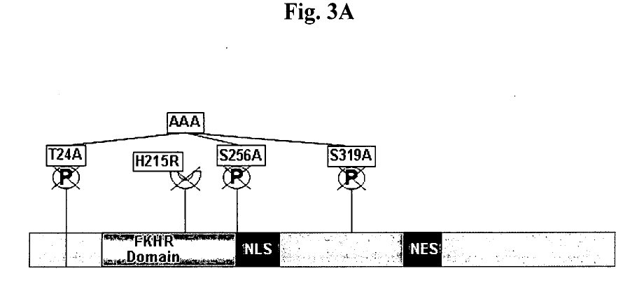

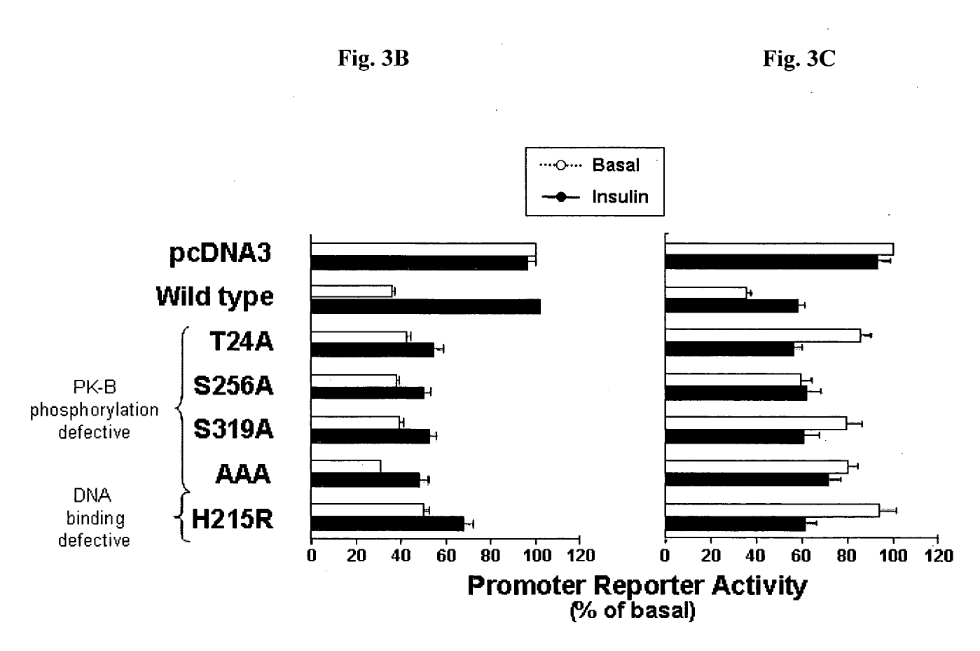

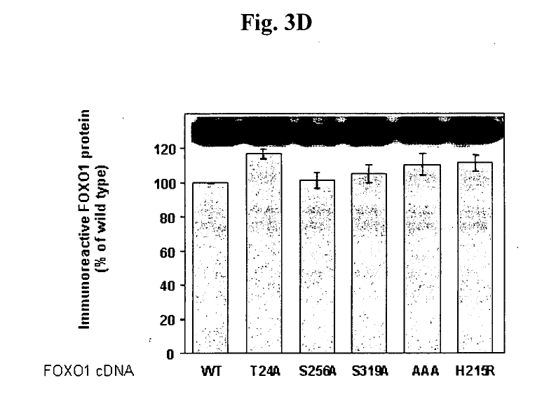

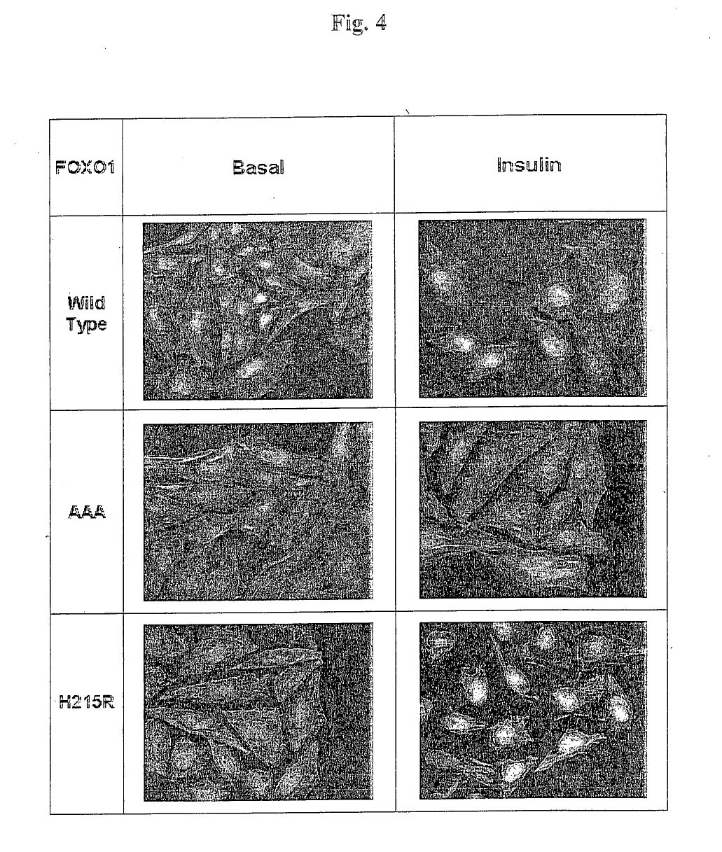

The differential contribution of the various functional domains of FOXO1 to PPARγ-P repression was studied under basal as well as insulin-mediated conditions using constructs that are point-mutated as schematically depicted in FIG. 3A. The contribution of each of the three PKB/Akt phosphorylation sites of FOXO1 was studied using non-phosphorylatable mutants of FOXQ1, T24A, S256A, S319A, and a triple mutant, AAA, in which all three phosphorylation sites were mutated to alanine. Cells were co-transfected with PPARγ promoter reporters along with the various FOXO1 mutants, and incubated either at basal conditions or with 100 mM insulin for 24 hrs. We found that mutations in each of the sites did not affect the basal capacity of FOXO1 to repress the PPARγ1 promoter, as revealed by similar basal promoter activity of the mutated proteins and the wild type protein (white bars, while significantly reducing insulin's derepression capacity, as shown by the lower activity of the mutant proteins as compared with the wild type protein (black bars) (FIG. 3B). All these mutants, however, exhibited either partial or complete loss of FOXO1 ability to repress PPARγ2-P (FIG. 3C).

We next used a DNA binding-defective mutant H215R, to study the contribution of the FOXO1 DNA binding domain (DBD; SEQ ID NO: 13). H215R mutation did not affect the basal capacity of FOXO1 to repress PPARγ1, while slightly reducing promoter derepression in presence of insulin (FIG. 3B). This mutant, however, completely lost its ability to repress the PPARγ2 promoter, and showed impaired responsiveness to insulin (FIG. 3C).

Western blot analysis performed on total cell lysates prepared from FOXO1-transfected cells, showed that all constructs used, wild type as well as mutants, are successfully expressed as immunoreactive FLAG-tagged FOXO1 proteins, to approximately the same extent (FIG. 3D), while mock-transfected primary rat adipocytes showed no FLAG-FOXO1 immunoreactivity (not shown).

To correlate between FOXO1 effects and its cellular localization, we studied the subcellular distribution of wild type FOXO1 and the various mutants in HEK-293 cells, under the same basal and insulin-mediated conditions (FIG. 4). HEK-293 cells were chosen for the immunofluorescent studies, as they easily stain to show more clearly the transfected proteins, and can serve as reasonable model to simulate FOXO1 translocation in adipocytes as they have been shown to express endogenous insulin signaling machinery, as well as endogenous FOXO1 (personal observation). In accordance with previous studies (Armoni et al., 2003), we found that in basal state wild type FOXO1 is distributed to the nucleus, and excluded from it upon insulin stimulation. Under both conditions the binding-defective mutant H215R behaved similarly to the wild type, while AAA mutant, which is irresponsive to PKB/Akt phosphorylation, was not excluded from nucleus in response to insulin.

### Example 4

### Cis-Elements on the PPARγ2 Promoter Mediate Its Regulation by FOXO1

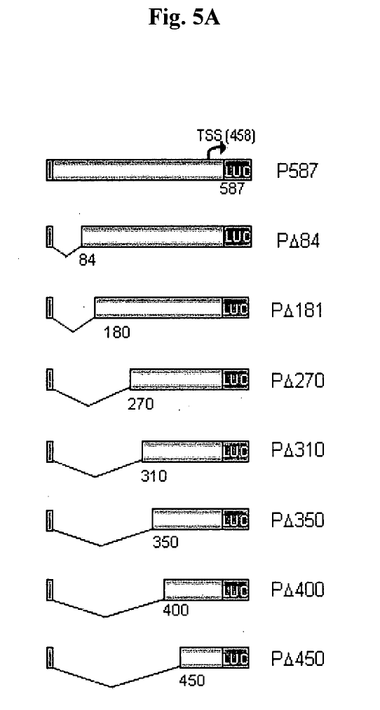

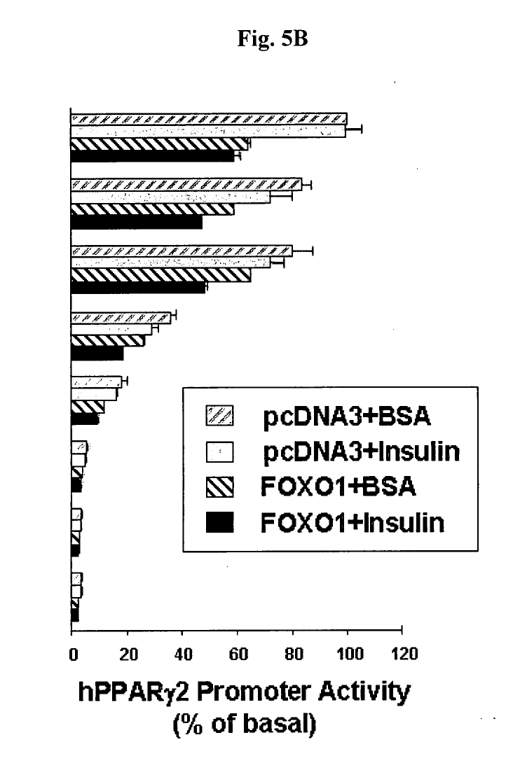

Our data indicate that the DNA binding domain of FOXO1 is crucial for the repression of PPARγ2, but not PPARγ1. Therefore we focused our efforts on identifying cis-elements in the PPARγ2 promoter that may serve as potential FOXO1 binding sites. We performed a progressive 5′-deletion analysis of the full-length PPARγ2 promoter reporter hG2-P587 (FIGS. 5A-5B). The 5′-deleted promoter reporters are shown in FIG. 5A. We found that deletion of bp 270 to 310 (SEQ ID NO: 4) in the promoter region led to a major depletion of FOXO1's ability to trans-repress the promoter in the absence of insulin, while not affecting insulin's ability to interfere with FOXO1 action. An additional bp 181-270 promoter region (SEQ ID NO: 24) also contributed to FOXO1 ability to repress the PPARγ2 promoter in either the presence or absence of insulin.

### Example 5

### FOXO1 Binding to the PPARγ2 Promoter

To investigate whether the regulation of PPARγ by FOXO1 involves a direct protein/DNA interaction, FLAG-tagged FOXO1 protein was translated in vitro and tested for the ability to bind the PPARγ2 promoter region containing bp 270-310 (SEQ ID NO: 4). Full-length FOXO1 protein was expressed at the expected size, as determined by SDS-PAGE analysis (FIG. 6A). Data from representative electromobility gel shift assay (EMSA) are shown in FIG. 6B. The addition of FOXO1 protein to the bp 270-310 DNA sequence led to the formation of a complex (indicated by the bold arrow on FIG. 6B). This binding is specific, as 100× and 200× fold molar excess of the unlabeled probe competed for binding in a dose-dependent manner. The specificity of this interaction is indicated by a supershift assay, showing retarded electrophoretic mobility of the FOXO1-DNA complex in the presence of specific anti-FOXO1 antibodies (indicated by the dotted arrow on FIG. 6B). The supershift was clearly induced in the presence of either an anti-FLAG monoclonal antibody or the anti-FOXO1 antibody H-128, which is directed against amino acids 471-598 near the carboxyl terminus of human FOXO1, but not in presence of anti-FOXO1 antibody N-18, which is directed against the FOXO1 amino terminus. This fact warrants further investigation as, beyond reflecting the quality of the antibody preparation, it may represent a differential role for the various FOXO1 domains in the interaction with the PPARγ gene promoter.

To find out whether FOXO1 binds to the human PPARγ2 promoter in cellulo, data obtained in EMSA were verified by chromatin immunoprecipitation (ChIP) assays. Human embryonal kidney (HEK)-293 cells were chosen to study FOXO1 binding to the human PPARγ2 promoter in its native form, i.e., within its native human chromatin context. FOXO1-transfected cells were subjected to two-step protein-DNA cross-linking. Lyzed cells were then sonicated and subjected to immunoprecipitation with either anti-FOXO1 antibody or a negative control IgG. DNA cross-linked to the immunoprecipitated FOXO1 was subjected to PCR using primers for the human PPARγ2 promoter (63-323 bp), or for the sequence encompassing 3×IRS in pGL2 as a positive control. As shown in FIG. 7A, data obtained using one-step cross-linking yielded no detectable PPARγ2 promoter signal in either anti-FOXO1 or negative control IgG immunoprecipitates; neither could we detect a PPARγ2 promoter signal in anti-FLAG immunoprecipitates (data not shown). Under these conditions, however, FOXO1 complexed with 3×IRS (FOXO1-3×IRS), used as positive control. Therefore, we have adopted ChIP assay using a two-step cross-linking procedure, as detailed by Nowak et al (2005). Data obtained using this two-step cross-linking showed that human PPARγ2 promoter is complexed with FOXO1 while PPARγ2 promoter signal is barely detected in negative control IgG immunoprecipitates (FIG. 7B). Similar results were obtained in cells that were transfected with FOXO1 and 3×IRS reporter (FOXO1-3×IRS), used as positive control. These data indicate that FOXO1 binds to native human PPARγ2 promoter in cellulo. Recapturing of the protein-DNA complexes, however, by ChIP assay, is available using a two-step rather then one-step cross-linking procedure.

### Example 6

### A Model for FOXO1 Repression of PPARγ1 and PPARγ2 Promoters and a New Paradigm to Increase Insulin Sensitivity in Adipocytes

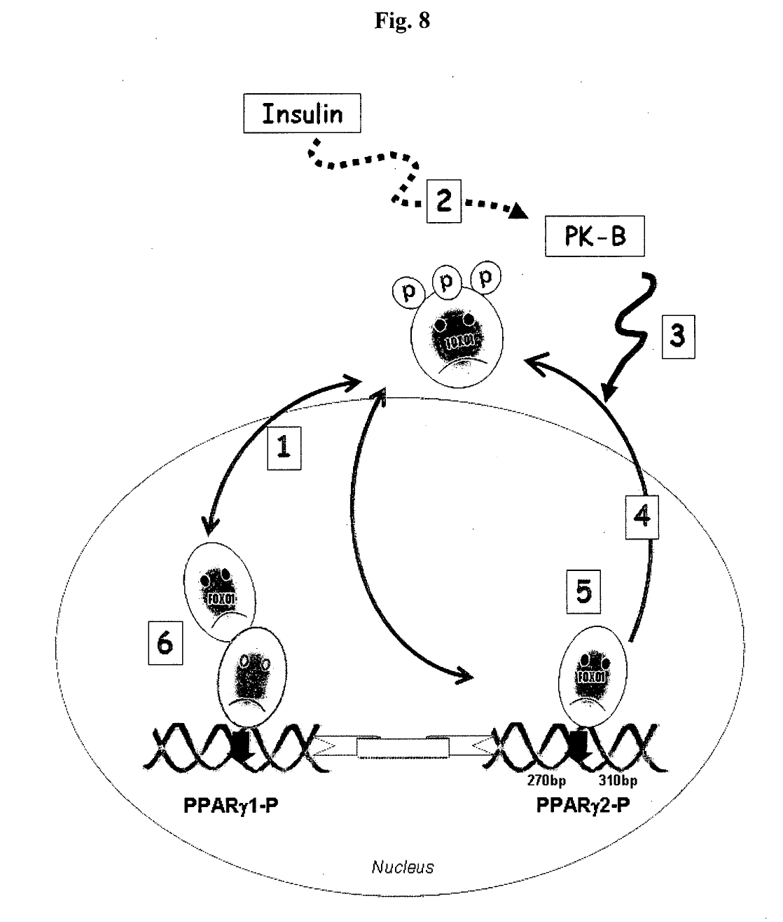

Based on the data we obtained from 5′-deletion, gel retardation, and ChIP analyses on the one hand, and FOXO1 mutation analysis on the other, we suggest the following model for FOXO1 repression of PPARγ1 and PPARγ2 promoters that is both tissue- and isoform-specific. According to this model, both promoters are repressed by FOXO1, however via a distinct mode of regulation, as only the repression of PPARγ2 promoter required an intact DBD of FOXO1, indicating a direct DNA-protein interaction. In accordance with that, we show that PPARγ2 promoter repression occurs via specific and direct binding of FOXO1 to defined DNA sequence on the promoter, both in vitro and in vivo. This newly-identified FOXO1 response element may thus serve as a molecular therapeutic target for the treatment of insulin resistance and type 2 diabetes. As for PPARγ1 regulation, several potential factors can mediate its regulation by FOXO1. Among those, most prominent are members of the PGC-1 family that have been previously shown to control the activity of PPARγ (see review of Corton and Brown-Borg, 2005). The model is shown in FIG. 8. 1) In the basal state FOXO1 recycles between the nucleus and the cytoplasm, but is localized mostly to the nucleus (see FIG. 4 and Tran et al., 2003). 2) Upon insulin stimulation, insulin signaling proceeds to PKB/Akt (Gilde and van Bilsen, 2003); 3) PKB/Akt activation leads to hierarchic phosphorylation of FOXO1 on three PKB/Akt consensus sites (depicted as circled P), T24, S256 and S319 (Fajas et al., 1997). 4) PKB/Akt phosphorylation of FOXO1 on either of these sites leads to its nuclear exclusion, followed by either complete or partial derepression of PPARγ1 or PPARγ2 promoter activities, respectively (see FIG. 2 for insulin effects, Armoni et al., 2003); 5) When in the nucleus, FOXO1 binds directly to the PPARγ2 promoter, via specific DNA sequence that encompasses 270-310 bp, but is probably extends beyond this region (as shown by ChIP). This leads to a dose-dependent repression of PPARγ2 transcriptional activity (represented by bold down-arrows). Mutations in either one of the PKB/Akt phosphorylation site, or in the FOXO1 DNA binding domain, that render FOXO1 protein either refractory to PKB/Akt, or defective in binding ability, respectively, lead to partial derepression of PPARγ2 promoter (Kaestner et al., 2000). FOXO1 also represses transcription from the PPARγ1 promoter. However, this effect probably does not include direct binding to the PPARγ1 promoter, as a H215R mutant that has defective binding capacity, still represses the promoter. Thus, repression of PPARγ1 promoter by FOXO1 probably involves an indirect regulation, maybe via some mediator factor. One promising candidate for this mediation is the PPARγ coactivator PGC-1, as it has been shown that Foxo1 regulate the activity and expression of PGC-1a, while PGC-1 family members interact with, and control the activity of PPARγ (Corton and Brown-Borg, 2005).

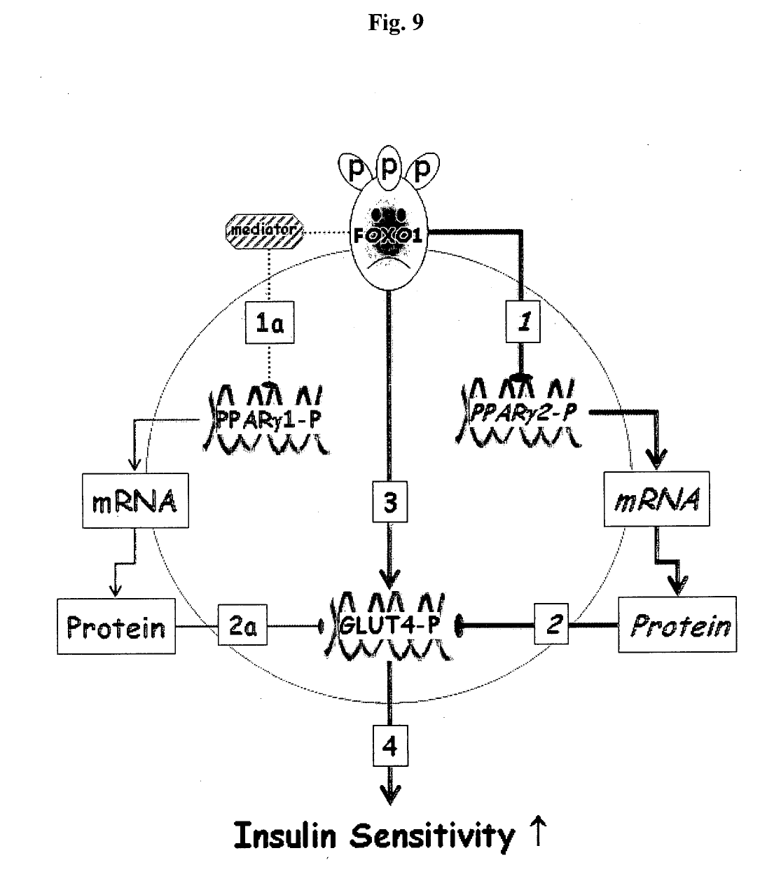

We introduce a novel paradigm to increase insulin sensitivity in adipocytes (summarized in FIG. 9). According to this paradigm: 1) FOXO1 represses of PPARγ gene expression directly (PPARγ2) or indirectly (PPARγ1); 2) As shown by us before both PPARγ1 and PPARγ2 proteins repress GLUT4 promoter activity; 3) Therefore, repression of PPARγ by FOXO1 leads to GLUT4 upregulation; 4) This subsequently results in enhanced glucose transport and cellular insulin sensitivity

### REFERENCES

Arden, K. C. (2004) Mol Cell 14(4), 416-418
Armoni, M., Harel, C., Bar-Yoseph, F., Milo, S., and Karnieli, E. (2005) J Biol Chem 280(41), 34786-34795
Armoni, M., Kritz, N., Harel, C., Bar-Yoseph, F., Chen, H., Quon, M. J., and Karnieli, E. (2003) J Biol Chem 278(33), 30614-30623.

Armoni, M., Quon, M. J., Maor, G., Avigad, S., Shapiro, D. N., Harel, C., Esposito, D., Goshen, Y., Yaniv, I., and Karnieli, E. (2002) J Clin Endocrinol Metab 87(11), 5312-5324.
Armoni M, Harel C, Karni S, Chen H, Bar-Yoseph F, Ver M R, Quon M J, Karnieli E. (2006) FOXO1 represses peroxisome proliferator-activated receptor-gamma1 and -gamma2 gene promoters in primary adipocytes. A novel paradigm to increase insulin sensitivity. J Biol Chem. 281:19881-91.
Bernardo A, Minghetti L. (2006) Curr Pharm Des., 12(1), 93-109.
Burgering, B. M., and Kops, G. J. (2002) Trends Biochem Sci 27(7), 352-360Corton, J. C., and Brown-Borg, H. M. (2005) J Gerontol A Biol Sci Med Sci 60(12), 1494-1509
Dowell, P., Otto, T. C., Adi, S., and Lane, M. D. (2003) J Biol Chem 278(46), 45485-45491
Fajas, L., Auboeuf, D., Raspe, E., Schoonjans, K., Lefebvre, A. M., Saladin, R., Najib, J., Laville, M., Fruchart, J. C., Deeb, S., Vidal-Puig, A., Flier, J., Briggs, M. R., Staels, B., Vidal, H., and Auwerx, J. (1997) J Biol Chem 272(30), 18779-18789
Fajas, L., Landsberg, R. L., Huss-Garcia, Y., Sardet, C., Lees, J. A., and Auwerx, J. (2002) Dev Cell 3(1), 39-49
Gilde, A. J., and Van Bilsen, M. (2003) Acta Physiol Scand 178(4), 425-434.
Kaestner, K. H., Knochel, W., and Martinez, D. E. (2000) Genes Dev 14(2), 142-146.
Kloting, N., Bluher, M., and Kloting, I. (2006) Diabetes Metab Res Rev 22(2), 146-154
Kurtz T. W. (2006) Am J. Med. 119(5 Suppl 1), S24-30
Li A. C., Palinski W. (2006) Annu Rev Pharmacol Toxicol, 46:1-39
Nakae, J., Biggs, W. H., 3rd, Kitamura, T., Cavenee, W. K., Wright, C. V., Arden, K. C., and Accili, D. (2002) Nat Genet 32(2), 245-253
Nakae, J., Kitamura, T., Kitamura, Y., Biggs, W. H., 3rd, Arden, K. C., and Accili, D. (2003) Dev Cell 4(1), 119-129
Nowak, D. E., Tian, B., and Brasier, A. R. (2005) Biotechniques 39(5), 715-725
Sambrook, J., Fritsh, E. F., and Maniatis, T. (1989) Molecular cloning: a laboratory manual, 2nd Ed Ed., Cold Spring Harbor, N.Y.
Shang, Y., Hu, X., DiRenzo, J., Lazar, M. A., and Brown, M. (2000) Cell 103(6), 843-852
Spiegelman, B. M., and Flier, J. S. (1996) Cell 87(3), 377-389
Tang, E. D., Nunez, G., Barr, F. G., and Guan, K. L. (1999) J Biol Chem 274(24), 16741-16746.
Tran, H., Brunet, A., Griffith, E. C., and Greenberg, M. E. (2003) Sci STKE 2003(172), RE5
Zhao, H. H., Herrera, R. E., Coronado-Heinsohn, E., Yang, M. C., Ludes-Meyers, J. H., Seybold-Tilson, K. J., Nawaz, Z., Yee, D., Barr, F. G., Diab, S. G., Brown, P. H., Fuqua, S. A., and Osborne, C. K. (2001) J Biol Chem 276(30), 27907-27912.

1. A method for detecting a modulator of transcription of a human PPAR gene promoter, comprising contacting a candidate compound with a cell transfected with an expression vector containing a heterologous gene operably linked to a PPAR promoter and an additional expression vector containing the FOXO1 gene encoding the FOXO1 protein, and comparing the level of expression of said heterologous gene in the presence of the compound and in the absence thereof, whereby a modulator of transcription of the human PPAR gene promoter is identified.

2. A method according to claim 1, wherein the PPAR gene promoter is a PPARγ gene promoter.

3. A method according to claim 2, wherein the PPARγ gene promoter is the PPARγ1 or PPARγ2 gene promoter.

4. A method according to claim 3, wherein said FOXO1 protein binds directly and specifically through its DNA binding domain to a DNA region within the PPARγ2 promoter and thus affects the transcription of the PPARγ2 gene.

5. A method according to claim 1, wherein the modulator represses the human PPARγ promoter and reduces the level of expression of the heterologous gene.

6. A method according to claim 1, wherein the modulator activates the human PPARγ promoter and increases the level of expression of the heterologous gene.

7. A method according to claim 4, wherein the DNA-binding domain of the FOXO1 protein binds to a sequence encompassing the 63 to 323 bp region of the human PPARγ2 promoter.

8. A method according to claim 7, wherein the DNA binding domain of the FOXO1 protein binds to a sequence encompassing the 270 to 310 bp region of the human PPARγ2 promoter.

9. A method according to claim 1, wherein the PPAR gene promoter is a PPARα or PPARβ\δ gene promoter.

10. A method according to claim 1, wherein the heterologous gene is a reporter gene.

11. The method according to claim 1, wherein said cell is an insulin-responsive cell.

12. The method according to claim 10, wherein said insulin responsive cell is an adipocyte, a smooth muscle cell, a skeletal muscle cell and a cardiac muscle cell.

13. The method according to claim 1, wherein said cell is a non-insulin responsive cell such as brain, liver, gut or pancreas cell.

14. A compound capable of modulating the FOXO1-mediated PPARγ1 or PPARγ2 gene expression.

15. A compound according to claim 14, which represses FOXO1-mediated PPARγ1 or PPARγ2 gene expression directly or indirectly.

16. A compound according to claim 14, which activates FOXO1-mediated PPARγ1 or PPARγ2 gene expression directly or indirectly.

17. A compound according to claim 14 which is a small organic molecule.

18. A compound according to claim 14 which is a peptide.

19. A compound according to claim 18, wherein the peptide is derived from the FOXO1 DNA-binding domain or an analog of said peptide.

20. A pharmaceutical composition for treatment, attenuation, or prevention of insulin resistance, type 2 diabetes and obesity, comprising a phammaceutically acceptable carrier and a compound according to claim 15.

21. A pharmaceutical composition for treatment, attenuation, or prevention of a human disease or disorder that is affected by activation of a PPARγ gene expression, comprising a pharmaceutically acceptable carrier and a compound according to claim 16.

22. A pharmaceutical composition according to claim 21, wherein said disease or disorder include atherosclerosis, coronary events, brain inflammation, and a neurodegenerative disease such as multiple sclerosis, Parkinson's disease and Alzheimer's disease.
